(19) United States
(12) Patent Application Publication
(10) Pub. No.: US 2005/0255038 A1
(43) Pub. Date: Nov. 17, 2005

(54) Conjugates comprising cancer cell specific ligands, a sugar and diagnostic agents and uses thereof

(75) Inventors: Michael F. Holick, Sudbury, MA (US); Halasya Ramanathan, Mysore (IN)

Correspondence Address: STERNE, KESSLER, GOLDSTEIN & FOX PLLC, 1100 NEW YORK AVENUE, N.W., WASHINGTON, DC, 20005, US

(73) Assignee: A and D Bioscience, Inc., Sudbury, MA (US)

(21) Appl. No.: 10/510,824

(22) PCT Filed: Apr. 14, 2003
(86) PCT No.: PCT/US03/11372

§ 371 Date: Oct. 12, 2004

Related U.S. Application Data
(60) Provisional application No. 60/371,672, filed on Apr. 12, 2002.

(51) Int. Cl. A61K 51/00; C07K 9/00

(52) U.S. Cl. 424/1.11; 530/322

(57) ABSTRACT

Disclosed are conjugates comprising cancer cell specific ligands, a sugar and diagnostic agents, and uses thereof, e.g. for imaging cancer cells and tumors in vivo.

### BACKGROUND OF THE INVENTION

1. Field of the Invention

The present invention relates to conjugates comprising a cancer cell specific ligand, linked to a sugar, linked to a diagnostic agent and uses thereof.

2. Related Art

In situ diagnostics of a live patient, e.g. with magnetic resonance imaging (MRI) or positron emission tomography (PET) to identify abnormalities due to disease onset and monitoring the progression of the ‘cure’ especially in the field of cancer and treatment, has been well known for years. This multi-disciplinary approach is still in the primitive stage due to the lack of specific reagents for targeting the cells of interest. However, ‘MRI and PET’ scans have grown very rapidly in solving the engineering aspects for better images. MRI uses gadolinium, manganese, B₁₀ and F₁₉ as contrast agents. Contrast agents containing coordinated or caged metal ions which have magnetic spins cannot be targeted to the site of interest. This is due to the fact that these diagnostics agents do not differentiate between the cancer cells and normal cells. PET scan involves imaging of the organs and the human body with radiolabeled isotopes present in organic molecules. Fluorine-18 isotope is used for this purpose in addition to many other radio labeled isotopes. Fluorinated organic molecules are of interest since the fluorine atom is similar to hydrogen and it is inert in the biological system.

Each one of the above techniques have their own advantages. For example, fluorinated organic molecules penetrate the blood brain barrier. In addition, MRI using gadolinium provides clear imaging using MRI. However, each technique suffers from the disadvantage of failing to target cells in the brain and other organs.

Similarly, problems associated with cancer chemotherapy include the high toxicity of the chemotherapeutic agents. This is a problem general to most of the chemotherapeutic agents including taxol, daunorubicin, cisplatin fluorouracil, vincristine, etoposide and others. Since cancer chemotherapeutic agents do not target cancer cells exclusively, toxicity cannot be avoided.

Toxicity is also apparent for other methods of treating cancer such as photodynamic therapy (PDT) and BNCT. In photodynamic therapy and boron neutron capture therapy, if the passive drug has no specificity, damage to the neighboring cells is inevitable when the drug is activated by either light or neutron capture. Similarly, diagnosis of cancer and monitoring the treatment progress of cancer patients using magnetic resonance imaging and contrast agents give poor target specificity.

Specific targeting of cancer cells has been attempted with antibody tethered liposomes. However, a problem with this approach is that the antibody should be tethered away from the huge liposomal molecule containing the cancer drug, so that the target specificity can be enhanced.

A large number of drugs and their metabolites are conjugated in the body as part of the elimination pathway. Glucuronic acid is the most frequent partner to the drug in conjugation. Remington's Pharmaceutical Sciences, A. Osol et al. (eds.), pp. 677 (1980).

ER (estrogen receptor), PR (progesterone receptor), and Her-2 receptors are over expressed in cancer cells. Peptides specific for ER have been prepared using a combinatorial peptide phage-display library for the preparation of ER-specific mAbs. S. Sompuram, et al., Clin. Chem. 48: 410-420 (2002).

The urokinase-type plasminogen activator (uPA) system is strongly linked to pathological processes, such as cell invasion and metastasis in cancer Dano K. et al., Adv. Cancer Res. 44:139-266 (1985)). Cancer cells exhibit up-regulation of uPAR, a cell surface receptor. Tapiovaara H. et al., Adv. Cancer Res. 69:101-33 (1996).

U.S. Pat. No. 6,120,765, discloses purified uPA peptides, having mitogenic activity and containing at least 6 amino acids of the EGF-like domain of uPA. These peptides bind to the urolinase plasminogen activator receptor (uPAR) on cells. Also described a conjugates between the uPA peptides and toxins, e.g. peptide toxins, for delivery to cells, e.g. epidermal cells.

U.S. Pat. No. 6,277,818 discloses uPAR targeting cyclic peptides comprising 11 amino acids corresponding to uPA (20-30), or substitution variants thereof. These cyclic peptides may be conjugated to diagnostic labels or therapeutic radionuclides. The compounds are reportedly useful for targeting uPAR expressed in pathological tissues, e.g. tumor cells. See also U.S. Pat. No. 5,942,492, which discloses cyclic peptides having 11 amino acids joined by a linking unit L, which are useful for inhibiting the growth or metastasis of cancerous tumors.

U.S. Pat. No. 6,258,360 discloses prodrugs of cytotoxic chemotherapeutic agents which are stable to endogenous mammalian enzymes and which are activated by tumor-specific antibody bonding and prodrug activation. The antibodies are capable of cleaving the protective moiety from the drug by esterase, amidase, hydrolase or glycosidase activity.

According to the present invention, by linking a cancer cell targeting agent to a sugar residue linked to a diagnostic agent containing phenolic, hydroxy, carboxyl or enolizable functional groups, one obtains a conjugate that offers many advantages. First and foremost, the cancer cell targeting agent (e.g the cyclic peptide) targets the cancer cell, thus directing the diagnostic agent to the target tissues and cells. The bond between the agent and sugar may then cleaved in situ to release the agent. In another embodiment, the diagnostic agent may be targeted to the “cadherin units” on cancer cells and thus be monitored by MRI/PET scanning techniques. The release of the diagnostic agent from the cancer cell may then be monitored over time. For example, when the sugar is a glucuronide, glucuronidases, which are more active on the surface of cancer cells compared to normal cells, will cleave the glucuronide bond, thus releasing the agent, and confirming the presence of cancer cells. Thus the conjugates of the invention have enhanced cancer cell target specificity.

### SUMMARY OF THE INVENTION

The invention relates to conjugates comprising a cancer cell specific ligand, linked to a sugar, linked to a diagnostic agent and uses thereof.

The present invention relates in particular to compounds of the Formula (I):
A-R′—X (I)
wherein A is the residue of a diagnostic label, R′ is a sugar residue, and X is a cancer cell specific ligand.

The invention also relates to pharmaceutical compositions comprising the compounds of the invention and a pharmaceutically acceptable carrier.

The invention also relates to a method of imaging cancer cells in vivo, comprising administering to an animal having cancer or suspected of having cancer a compound of the invention, and detecting the diagnostic agent, e.g. by imaging the patient using planar or SPECT gamma scintigraphy, positron emission tomography, or magnetic resonance.

The invention also relates to a method of preparing a compound of Formula (I) which comprises condensing a protected sugar with a diagnostic agent. The conjugate is then condensed with a cancer cell specific ligand and the protecting groups may then be partially or completely removed.

### DETAILED DESCRIPTION OF THE INVENTION

Cancer Cell Specific Ligands

These ligands include small organic molecules, carbohydrates and peptides that bind specifically to cancer cells. Preferably, the ligands are peptides, preferably 5-8 amino acids long within a peptide comprising 20 amino acids or less and, more preferably, are cyclic peptides of 20 amino acids or less and containing the ligand peptide of 5-8 amino acids. Also preferably, the ligand portion of the cyclic peptide are devoid of a lysine unit, eliminating the possibility that the side chain amine will interfere with the new amide bond formation with the sugar-agent/label conjugate. One such peptide which contains a lysine end unit bearing free amine ready for linkage to a pro-drug is ER Peptide 3: Acetyl-D F Q C P Y V E C V V N A P G G K-NH2 (SEQ ID NO:1). Preferably, the peptide is a cyclic peptide having a disulfide bond between the two cysteine groups. Another example is Asp-Phe-Gln-Cys-Pro-Tyr-Val-Glu-Cys-Val-Val-Asn-Ala-Pro-Gly-Gly-Lys-NH2 (SEQ ID NO:2).

uPA (urokine plasminogen activator), which converts plasminogen to plasmin, is more widely expressed in the cancer cells. U.S. Pat. No. 6,120,765 discloses uPA peptide fragments having more than 5 amino acids and less than 13 contiguous amino acids and bind to the urokinase plasminogen activator receptor (uPAR). These peptides have mitogenic action due to the agonist action of uPA. One such cyclic protein useful in the present invention is shown below and has a free carboxyl end for attachment to amine end of prodrugs. Any of the peptides described in U.S. Pat. No. 6,120,765, whether cyclic or straight chained, may be used in the practice of the invention.

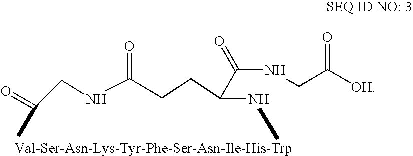

A synthetic variant useful in the practice of the present invention that accommodates attachment to carboxyl end of a glucuronate is also described in U.S. Pat. No. 6,120,765:

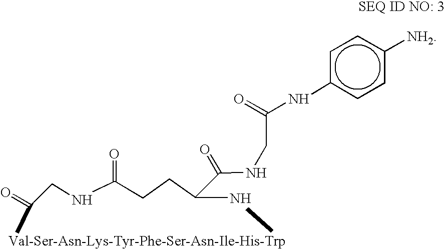

Other examples of peptides that may be used in the practice of the invention include the uPAR-targeting peptide of 11 amino acids, and substitution variants thereof, corresponding to human uPA(20-30), as described in U.S. Pat. No. 6,277,818.

Additional examples of peptides that may be used in the practice of the invention are described in U.S. Pat. No. 6,339,139 which describes peptide conjugates for targeting cancer cells. According to this patent, IGF-I R and IGF-II R are over-expressed in human hepatic cancer and other malignancies. EGF R is highly expressed in human hepatic, mammary, ovarian, gastric, cervix cancer, glioblastoma, lung adenocarcinoma, nasopharyngeal cancer etc. VEGF R is over-expressed in vascular endothelial cells of tumor blood vessels and some cancer cells.

Additional examples of peptides that may be used are disclosed in U.S. Pat. No. 6,087,109 which discloses ST receptor proteins. Such ST receptors are found on colorectal cells, including local and metastasized colorectal cancer cells. In normal human individuals, ST receptors are found exclusively in cells of intestine, in particular in cells in the duodenum, small intestine (jejunum and ileum), the large intestine, colon (cecum, ascending colon, transverse colon, descending colon and sigmoid colon) and rectum. Thus, conjugates of the present invention comprising peptide ligands for the ST receptor may be used to treat local and metastasized colorectal cancer.

Small peptides are especially preferred, as they are easy to use in organic synthesis for conjugation and targeting. Cyclic peptides are also useful since the shape of the cyclic peptides don't change or are perturbed by the neighboring diagnostic agent attachment.

Where the peptide contains an internal lysine, it is preferably protected prior to the amide bond formation with the sugar. Examples of lysine protecting groups include acetyl groups, t-butyloxycarbonyl (BOC) and benzyloxycarbonyl groups (carbamate protection). BOC and benzyloxycarbonyl groups can be deprotected with trifluroacetic acid or hydrogenation, respectively, depending upon compatibility with the sugar/agent. The acetyl groups may be left without deprotection.

Other cancer cell specific ligands include those described in U.S. Pat. No. 6,322,770 which discloses targeting moiety the targeting moiety comprising an indazole that binds to a receptor that is expressed in tumor neovasculature.

Additional cancer cell specific ligands are disclosed in U.S. Pat. No. 5,942,492, which discloses compounds having affinity for certain cancer cells, e.g. lung carcinomas, colon carcinomas, renal carcinomas, prostate carcinomas, breast carcinomas, malignant melanomas, gliomas, neuroblastomas and pheochromocytomas.

Other cancer cell specific ligands include monoclonal antibodies and fragments thereof that bind to cancer cell surface proteins, e.g. receptors. Examples of such antibodies include those specific for the extracellular domain of the EGF receptor as disclosed in U.S. Pat. No. 6,217,866.

Other cancer cell specific ligands include cyclic peptides which recognize glucuronidase, uPA, ER (see S. Sompuram, et al., Clin. Chem. 48: 410-420 (2002)), PR (progesterone receptor), and Her-2 receptors.

Diagnostic Agents

Examples of diagnostic agents which may be linked to the sugar residue include crown ethers which bind metals such as gadolinium, an MRI contrast agent. Other diagnostic labels include positron-emission tomography (PET) labels using fluoroglycose conjugated drug tethered with one of the above mentioned cancer cell specific ligands. In another embodiment, the diagnostic label is an organic molecule labeled with ¹⁸F. Moresco, R. M. et al., Nucl. Med. Commun. 22(4):399-404 (2001); Sharma M. et al., Carbohydr. Res. 198(2):205-21 (2001).

Sugar Residues

Sugar residues that are useful in the practice of the present invention include glucose, glucosamine, glucuronic acid, ribose, and the 2-deoxy derivatives thereof, e.g. 2-deoxy glucose, 2-deoxy-2-fluoroglucose and 2-deoxy ribose. In a preferred embodiment, the sugar residue is glucose which renders the conjugate more polar and water soluble, thus ameliorating any solubility problems of the cancer cell specific ligand and/or agent.

Other sugar residues that may be used in the practice of the invention include derivatives of glucose, glucosamine and glucuronic acid. Preferably, endogenous glucosidases, glucuronidases, and amidases will recognize and cleave the sugar derivative-agent bond, thus releasing the agent. In another embodiment, the diagnostic agent may be targeted to the “cadherin units” on cancer cells and thus be monitored by MRI/PET scanning techniques. The release of the diagnostic agent from the cancer cell may then be monitored over time. In other embodiments, the derivative is chosen so that the sugar derivative-agent bond is not cleaved, thus retaining the agent on or in the cancer cells. Particular examples of such derivatives include the 2-fluoro derivatives, e.g. 2-fluoroglucose and 2-fluoroglucuronic acid. Preferably, the fluorine is the radioisotope ¹⁸F. More preferably, the derivative is 2-fluoro¹⁸-2-deoxy glucose (i.e. 2-¹⁸fluoro glucose). Such ¹⁸F derivatives may be monitored by PET scan, thus allowing diagnosis and cure at the same time. Methods of making 2-¹⁸fluoro-2-deoxyglucose are described in U.S. Pat No. 5,436,325.

An example of a compound of the present invention having bearing ¹⁸F has the formula:

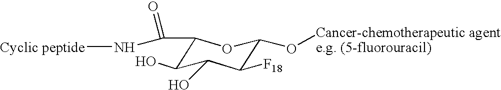

The sugar residues may have free hydroxy groups, or the hydroxy groups may be acylated, e.g. with a group R₄(C═O)—, wherein R₄ is hydrogen, C₁₋₆ alkyl, C₆₋₁₀ substituted or unsubstituted aryl or C₇₋₁₆ aralkyl. Preferably, the acyl groups are acetyl or propionyl. Other preferred R₄ groups are phenyl, nitrophenyl, halophenyl, lower alkyl substituted phenyl, lower alkoxy substituted phenyl and the like or benzyl, lower alkoxy substituted benzyl and the like.

The sugar residues may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

The compound of this invention can form an acid/base addition salt with an inorganic or organic.

Methods of Making the Compounds of the Invention

The cancer cell specific ligands may be conjugated to the diagnostic agent by the addition of a sugar using protected glycose and glucuronides with activated anomeric center such as halo, trichloro imidate, thiophenyl and their sulfoxides as is well known in the art. Bifunctional units like glucuronic acid and glucosamine are preferred as they are stable molecules and are known to be cleaved by glucuronidase and glucosidase enzymes and also the cyclic peptide can be added to the remaining bi-functionality namely the amine or carboxylic acid groups. Glucuronidase enzymes are more prevalent and highly expressed in cancer cells over normal cells and thus glucuronidation of the diagnostic agent is preferred such that the targeting cyclic peptide with the free amine end can be amidated later.

Amadori rearrangement is a main concern when linking sugars with peptides. This reaction is also called Malliard reaction. However, the blocking of the anomeric position of the sugar avoids this adverse reaction between the sugar and the peptide. The anomeric position may be blocked with the agent having a hydroxy function (e.g. alcohols/phenols/carboxyl groups/enols) present on the agent The coupling of the agent with a cancer cell specific ligand such as a peptide can be carried out with reagents such as EDC and DMT-MM {4-(4,6-dimethoxy-1,3,5-triazin-2-yl)-4-methylmorpholinium chloride}. DMT-MM is more versatile than EDC, as the conjugation reaction may be carried out in protic solvents such as methanol, ethanol and water. Amide bond formation may be accomplished elegantly by this method.

Glucuronate/agent conjugates may be prepared by reacting protected glucuronic acid containing an activated anomeric position (e.g. the 1-halo, trimethylsilyl and tricholoroimidate derivatives) together with and the agent containing hydroxyl, phenolic and carboxyl functions. After the conjugation and deprotection, the carboxyl group of the glucuronate may be conjugated to the amine end of a cancer cell specific ligand such as a peptide. Similarly protected glucosamine may be conjugated and deprotected to give a conjugate having a free amine that may be conjugated to the C-terminal end of a peptide. The amide bond may be obtained by reaction with DMT-MM in a protic solvent such as methanol and water in which the peptide and glucuronates are soluble. Preferably, the cancer cell specific ligand is a small cyclic peptide, e.g. a 5-30 amino acid peptide.

In one particular embodiment, the agent may be conjugated to glucosamine as shown in Scheme 1:

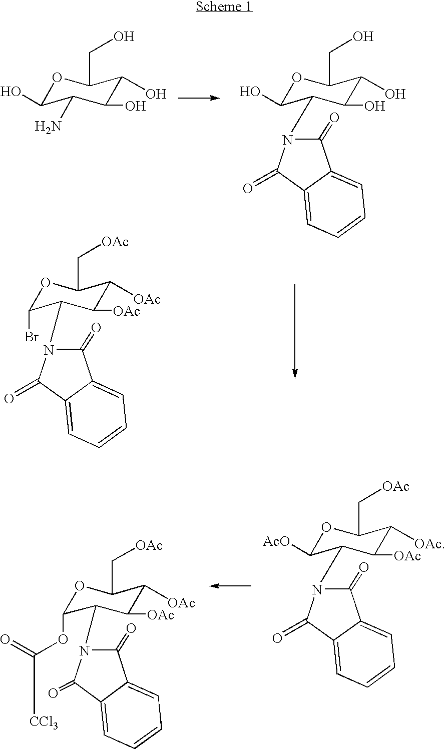

In place of phthalamide, the protecting group may also be the p-anisaldehyde imine which is easier to deprotect under mildly acidic conditions.

In the next step, amide formation reaction is carried out with a cancer cell specific ligand, in particular, peptides chosen from cyclic peptides of uPA-agonists. This reaction is carried out after deprotection and reacting either the amine or acid of the sugar with DMT-MM and the peptide that has an exposed carboxyl or amine function, respectively.

A number of sugar-diagnostic agent conjugates are shown in Scheme 2.

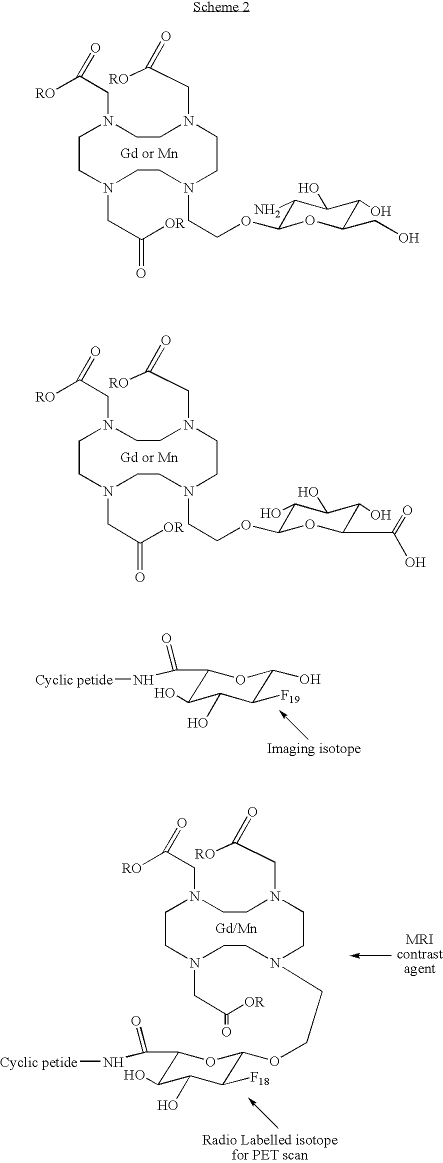

Similarly, glucuronide conjugates may be prepared as shown in Scheme 3.

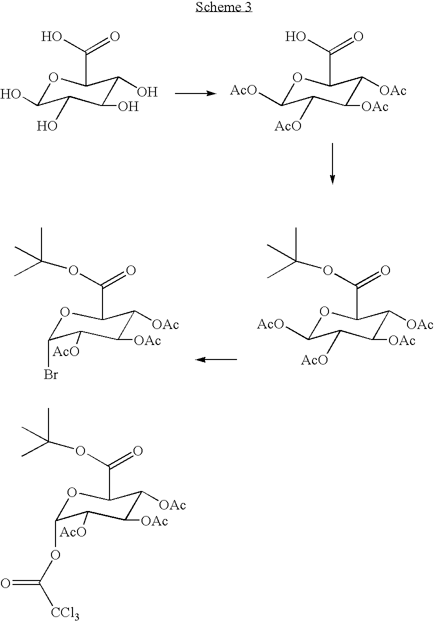

Other glucuronide conjugates are shown in Scheme 4.

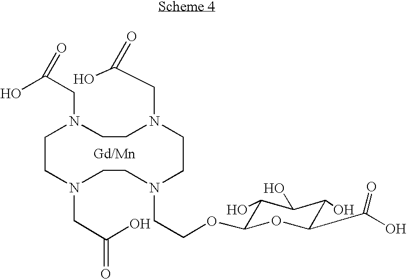

Examples of conjugates comprising uPA derived cyclic peptide and various sugars and cancer chemotherapeutic agents include:

Examples of conjugates comprising uPA derived cyclic peptide and various sugars and Gadolinium doped contrast agents include (SEQ ID NO:3 for each sequence):

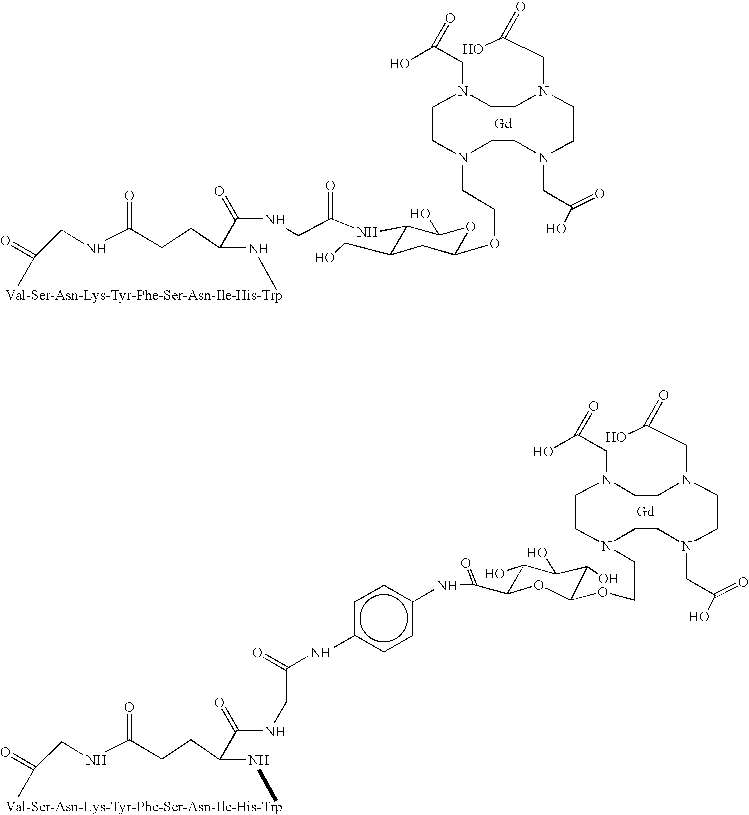

The amide bond formation can be prepared after de-protection and reacting either the amine or acid of the sugar with DMT-MM and the peptide that has an exposed carboxyl or amine function, respectively.

Methods of Use and Formulation

The binding of the conjugate in vivo can be monitored by utilizing imaging techniques, e.g. with gadolinium doped small molecules conjugated with the peptides, thus establishing the site specificity in compared to the corresponding untargeted molecule not containing the peptide. If the sugar is cleaved by glucuronidases expressed on the surface of cancer cells, one may monitor the release of the diagnostic agent, thus confirming the presence of cancer cells.

Also once the presence of cancer is confirmed, one may then replace the diagnostic agent with a cancer chemotherapeutic agent to kill the cancer cells. In the case of fluorine containing glycose moiety, the progression of the treatment can be monitored by having fluorine 18 labels. Being a small molecule, the conjugate can be monitored by standard assay techniques of HPLC, LC-MS etc. unlike huge antibodies attached to the cancer drugs.

Particularly preferred routes of administration of the compounds of the present invention are per os, such as elixirs, tablets and capsules, as exemplified below, and by i.v. administration.

More generally, the compounds of the present invention can be administered in any appropriate pharmaceutically acceptable carrier for oral administration. The compounds of the invention may also be administered in any appropriate pharmaceutical carrier for parenteral, intramuscular, transdermal, intranasal, buccal or inhalation administration. They can be administered by any means that allow them to reach the target cells and tissues.

The dosage administered will depend on the age, health and weight of the recipient, the nature of the cancer, and the kind of concurrent treatment. An exemplary systemic dosage is about 0.1 mg to about 500 mg. Normally, from about 1.0 mg to 100 mg daily of the compounds, in one or more dosages before the diagnostic procedure, is effective to obtain the desired results. One of ordinary skill in the art can determine the optimal dosages and concentrations of active compounds with only routine experimentation.

The compounds can be employed in dosage forms such as tablets and capsules for oral administration. Such dosage forms may comprise well known pharmaceutically acceptable carriers and excipients. In a preferred embodiment, the dosage forms comprise cyclodextran and/or other saccharides and/or sugar alcohols. The compounds may also be formulated in a sterile liquid for formulations such as solutions (e.g. in saline) or suspensions for parenteral use. A lipid vehicle can be used in parenteral administration. The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Remington's Pharmaceutical Sciences, 18^{th} Edition, Gennaro et al. (eds.), 1990, exemplifies methods of preparing pharmaceutical compositions.

The compounds may also be employed in fast dissolving dosage forms, as described in U.S. Pat. No. 6,316,027, comprising the compounds of the invention, water, gelatin and other ingredients.

The compounds of the invention may be formulated as part of a limposomal composition.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, drops or transdermal patch. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight of the active compounds.

The compounds of the invention are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, veterinary animals and pets, although the invention is not intended to be so limited.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

1. A compound comprising a cancer cell specific ligand, linked to a sugar, linked to a diagnostic agent.

2. The compound of claim 1, wherein said cancer cell specific ligand is a peptide.

3. The compound of claim 2, wherein said peptide is a cyclic peptide of 20 amino acids or less and containing the ligand peptide of 5-8 amino acids.

4. The compound of claim 2, wherein said peptide is a uPA agonist.

5. The compound of claim 1, wherein said sugar is glucose, glucosamine, glucuronic acid, ribose, or the 2-deoxy derivatives thereof.

6. The compound of claim 1, wherein said agent is a diagnostic agent comprising ¹⁸F.

7. The compound of claim 1, wherein said agent is 2-¹⁸F-2-deoxyglucose.

8. The compound of claim 1, which has the formula (SEQ ID NO:3 for each sequence):

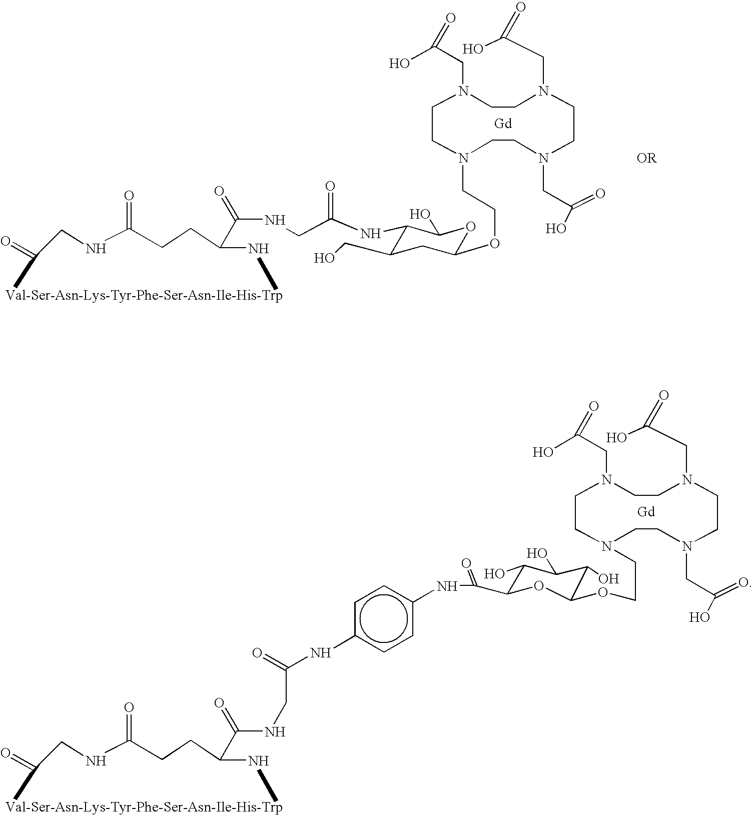

9. A pharmaceutical composition comprising the compound of claim 1 and a pharmaceutically acceptable carrier.

10. A method of imaging cancer in vivo, comprising administering to an animal having cancer or suspected of having cancer a compound of claim 1, and detecting the diagnostic agent.


SEQUENCE LISTING (3 sequences)

SEQ ID NO: 1 - protein, 17 aa, Artificial Sequence
  ER Peptide 3
DFQCPYVECVVNAPGGK

SEQ ID NO: 2 - protein, 17 aa, Artificial Sequence
  Synthetic Peptide
DFQCPYVECVVNAPGGK

SEQ ID NO: 3 - protein, 11 aa, Artificial Sequence
  Synthetic Peptide
VSNKYFSNIHW